(19) United States
(12) Patent Application Publication
(10) Pub. No.: US 2016/0310380 A1
(43) Pub. Date: Oct. 27, 2016

(54) Personal Lubricant Containing Volatile Fatty Acids

(71) Applicant: Michael David Bergsma, Carlsbad, CA (US)

(72) Inventor: Michael David Bergsma, Carlsbad, CA (US)

(21) Appl. No.: 15/136,161

(22) Filed: Apr. 22, 2016

Related U.S. Application Data
(63) Continuation of application No. 62/152,569, filed on Apr. 24, 2015.

(51) Int. Cl. A61K 8/36; A61Q 19/00

(52) U.S. Cl. CPC A61K 8/36; A61Q 19/00; A61K 2800/59

(57) ABSTRACT

An unique concentration and blend of volatile fatty acids provides an odor, smell, and taste when included in a composition with other ingredients, that includes cosmetic and personal use products, such as lubricants, body spays, perfumes and creams. The invention retains specifically to a personal lubricant containing volatile fatty acids for enhancing sexual intimacy between partners prior to, during or after intercourse, by providing small concentrations of volatile fatty acids that can be detected as odor, taste or scent, of which are already know to be present in real vaginal secretions, additionally to provide lubrication that decreases the wall surface frictional coefficients in the vagina, which increases the intimacy between sexual partners, and increases the retention of moisture in the vagina, or alternatively to be used to lubricate adult sexual toys, providing a fluid that resembles real vaginal secretions in odor and scent. The personal lubricant containing volatile fatty acids generally includes volatile fatty acids, gel/lubricant, moisturizer, pH adjuster, preservative, flavors, and sweetener.

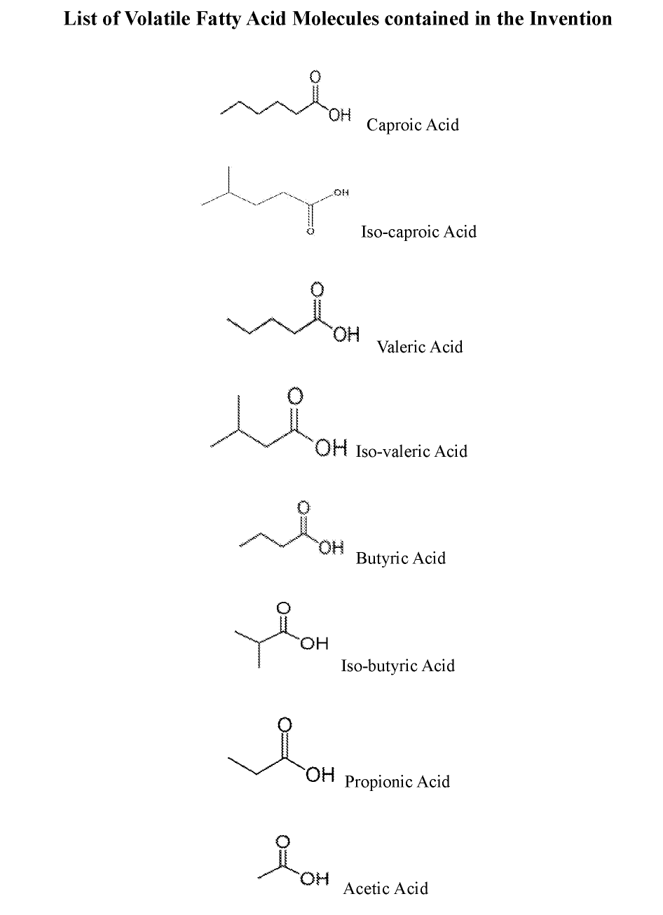

### BACKGROUND OF THE INVENTION

The present invention relates generally to cosmetic and personal use products, such as lubricants, body spays, perfumes and creams, and more specifically it relates to personal lubricants. In every embodiment of the invention, there contains an unique concentration and blend of volatile fatty acids, which purpose is to provide one or more components of odor, taste, smell, or exfoliation. In the embodiment of a personal lubricant, the invention serves to enhance sexual perceptions and sexual intimacy between partners prior to, during or after intercourse, by providing specific concentrations of volatile fatty acids, of which are already know to be present in human secretions, including but not limited to vaginal secretions, of which can be detected as odor, scent and taste, and by also providing the lubrication that decreases the wall surface frictional coefficients in the vagina, which increases the intimacy between sexual partners, and increases the retention of moisture in the vagina, or alternatively to be used to lubricate adult sexual toys, providing a fluid that resembles real vaginal secretions in odor, scent, taste, and lubrication. In the embodiment of a body spray or perfume, it can be applied in areas of the body which are already natural sources of volatile fatty acids. In the embodiment of a cream, small concentrations of volatile fatty acids provide a gentle exfoliator for use in skin care.

### BRIEF SUMMARY OF THE INVENTION

The invention generally relates to cosmetic and personal use products, such as lubricants, body spays, perfumes and creams. In every embodiment of the invention, a unique concentration and blend of volatile fatty acids is present, to provide odor, smell, taste, or exfoliant properties. In the embodiment of a personal lubricant, which is the main embodiment described in this document, the blend of volatile fatty acids is a component of the personal lubricant as a whole. This invention describes both the composition of the lubricant and the process for creating it.

There has thus been outlined, rather broadly, some of the features of the invention in order that the detailed description thereof may be better understood, and in order that the present contribution to the art may be better appreciated. There are additional features of the invention that will be described hereinafter.

In this respect, before explaining at least one embodiment of the invention in detail, it is to be understood that the invention is not limited in its application to the details of construction or to the arrangements of the components set forth in the following description or illustrated in the drawings. The invention is capable of other embodiment's and of being practiced and carried out in various ways. Also, it is to be understood that the phraseology and terminology employed herein are for the purpose of the description and should not be regarded as limiting.

An object is to provide a personal lubricant containing volatile fatty acids for enhancing sexual intimacy between partners prior to, during or after intercourse, by providing small concentrations of volatile fatty acids that can be detected as odor or scent, of which are already know to be present in real vaginal secretions, additionally to provide lubrication that decreases the wall surface frictional coefficients in the vagina, which increases the intimacy between sexual partners, and increases the retention of moisture in the vagina, or alternatively to be used to lubricate adult sexual toys, providing a fluid that resembles real vaginal secretions in odor, scent and taste.

Another object is to provide a personal lubricant containing volatile fatty acids that provides volatile odors, tastes and scents. Volatile fatty acids include acetic acid, propionic acid, butyric acid, iso-butyric acid, valeric acid, iso-valeric acid, caproic acid, and iso-caproic acid. Volatile Fatty Acids are present in human vaginal secretions in the order of 0.001% acetic acid, 0.0007% propionic acid, 0.00005% iso-butyric acid, 0.00065% butyric acid, 0.0002% iso-valeric acid, 0.00005% iso-caproic acid. The concentrations of volatile fatty acids in the lubricant composition described in this invention is from 1 to 100 times the naturally occurring concentrations. The ratios of concentrations of the volatile fatty acids is important to obtain an odor and scent that has a pleasing smell, and that smell can be either weak, moderate, or strong, depending on personal tastes.

Another object is to provide a personal lubricant containing volatile fatty acids that gels and lubricates. The composition of the lubricant is a gelling compound containing ingredients as such as carrageenan, sodium carbomer, hydroxyethyl methyl cellulose, hydroxyethyl cellulose, propylene glycol (PEG), PEG-3350, glycerin. The resulting compositions decrease the wall surface frictional coefficients in the vagina, increase the intimacy between sexual partners, and increase the retention of moisture in the vagina. The viscosity range is between 200 and 30,000 centipoise, more preferably at 5,500 centipoise.

Another object is to provide a Personal Lubricant Containing Volatile Fatty Acids that moisturizes. The composition of the lubricant/gel contains water as the moisturizer.

Another object is to provide a Personal Lubricant Containing Volatile Fatty Acids that contains a pH adjuster, such as the Potassium or Sodium salts of Acetic Acid or Citric Acid. The ratio of the concentrations of the alkaline salt (Potassium hydroxide or Sodium Hydroxide with the aliphatic acid (Citric Acid or Acetic Acid) creates an chemical equilibrium that serves to buffer the pH towards a neutral range.

Another object is to provide a Personal Lubricant Containing Volatile Fatty Acids that contains a preservative, such as methylparaben, propylparaben, or sodium benzoate.

Another object is to provide a Personal Lubricant Containing Volatile Fatty Acids that contains flavors, such as provided by citric acid, natural flavors or synthetic flavors.

Another object is to provide a Personal Lubricant Containing Volatile Fatty Acids that contains sweeteners, such as provided by sorbitol, glycerin, xylitol, or sugar alcohols.

In the embodiments of the disclosure, the following is applicable, The lubricant/gel may consist of one or more of, but not limited to
sodium carbomer, silicone, dimethicone, ethylmethylcellulose, methylcellulose, carrageenan, glycerin, polyhidritic glycerols, carboxymethylcellulose, guar gums, propylene glycol, alginates, PEG-3350.
aliphatic acids with 1, 2, 3, 4, 5, 6, 7 carbon atoms in the chain, straight (N-) or branched (iso-).
lavender oil, clove oil, musk oil, rose oil, sandalwood oil, jasmine oil, cinnamon oil, nutmeg oil
preservatives as needed or required by the CFR-21, methylparaben, propylparaben, sorbate, sodium hydroxymethylglycinate, sodium benzoate
citric acid, hydrogen peroxide, lactic acid, sodium hydroxide, potassium hydroxide
sweeteners as needed, sorbitol, xylitol, glycerin, sugar alcohols
flavors as needed, citric acid, natural or synthetic flavors

The resulting short chain fatty acids lubricant/gel composition may be scented or unscented, with color or colorless, flavored or flavorless, water-based, may include humectants like glycerin and surfactants as needed. The vaginal formula is preferably pH balanced with a range between 3.2 and 10.6, more preferably at 4.0. The viscosity range is between 200 and 30,000 centipoise, more preferably at 5,500 centipoise.

Other objects and advantages of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein the List of Volatile Fatty Acid Molecules contained in the Invention is:

Caproic Acid

Iso-caproic Acid

Valeric Acid

Iso-valeric Acid

Butyric Acid

Iso-butyric Acid

Propionic Acid

Acetic Acid

### DETAILED DESCRIPTION OF THE INVENTION

### A. Overview

Turning now descriptively to the drawings, in which similar reference characters denote similar elements throughout the several views, the figures illustrate volatile fatty acids. Descriptions of the lubricant/gel, moisturizer, pH adjuster, preservative, flavors, and sweetener are do not have associated drawings as these are conventional main elements.

### B. Novel Main Element: Volatile Fatty Acids

A blend of acetic acid, propionic acid, butyric acid, iso-butyric acid, valeric acid, iso-valeric acid, caproic acid, and iso-caproic acid. The blend of fatty acids is volatile, and functions to provide a volatile odor and scent.

These are both short-chain fatty acids with aliphatic tails of less than six carbon atoms and medium-chain fatty acids with aliphatic tails of less than 7 atoms, including Acetic Acid, Propionic Acid, Butyric Acid, Iso-butyric acid, Valeric Acid, Iso-valeric Acid, Caproic Acid, Iso-caproic Acid.

Personal lubricants that provide lubrication during sexual intercourse are known. However, personal lubricants utilizing volatile fatty acids to increase sexual intimacy are not known.

Several studies have shown how odor and scent may increase sexual intimacy between partners when in close proximity. For example, essential oils often used in aromatherapy and other odorants have also been shown to increase sexual intimacy between partners in close proximity. In the same manner volatile fatty acids may increase sexual intimacy between partners when in close proximity, as these components already occur naturally in vaginal secretions.

In producing the liquid compositions of the present disclosure, there are preferred amounts of volatile fatty acids. More particularly, it is preferred to use the following ingredients in the ranges of:

Acetic Acid 0.01% to 0.15% (w/w)

Propionic Acid 0.01% to 0.05% (w/w)

Butyric Acid 0.00065% to 0.00800% (w/w)

Iso-butyric Acid 0.00025% to 0.00600% (w/w)

Valeric Acid 0.0002% to 0.0040% (w/w)

Iso-valeric Acid 0.0002% to 0.0040% (w/w)

Iso-caproic Acid: 0.00005% to 0.00200% (w/w)

Caproic Acid 0.00005% to 0.00200% (w/w)

In one embodiment, which is a preferred embodiment, the percentage weight (w/w) of the volatile fatty acid ingredients in the lubricant is:

Acetic Acid 0.15% (w/w)

Propionic Acid 0.05% (w/w)

Butyric Acid 0.008% (w/w)

Iso-butyric Acid 0.006% (w/w)

Iso-valeric Acid 0.004% (w/w)

Caproic Acid 0.002% (w/w)

In another embodiment, the percentage weight (w/w) of the ingredients in the lubricant is:

Acetic Acid 0.15% (w/w)

Propionic Acid 0.05% (w/w)

Butyric Acid 0.004% (w/w)

Iso-butyric Acid 0.003% (w/w)

Iso-valeric Acid 0.002% (w/w)

Iso-caproic Acid 0.001% (w/w)

In another embodiment, the percentage weight (w/w) of the ingredients in the lubricant is:

Acetic Acid 0.015% (w/w)

Propionic Acid 0.005% (w/w)

Butyric Acid 0.002% (w/w)

Iso-butyric Acid 0.0015% (w/w)

Iso-valeric Acid 0.001% (w/w)

Iso-caproic Acid 0.0005% (w/w)

### C. Conventional Main Element: Gel/Lubricant

The gel is a blend of ingredients that may include the following: water, sodium carbomer, carrageenan, polyethylene glycol (PEG), PEG-3350, glycerin.

Fluid friction occurs between layers within a fluid that are moving relative to each other. This internal resistance to flow is described as viscosity The lubricant/gel has a viscosity range between 200-30,000 centipoise, preferably in the order of 5,500 centipoise.

In producing the liquid compositions of the present disclosure, there are preferred amounts of Gel/Lubricant compounds. More particularly, it is preferred to use the following ingredients in the ranges of:

Water 25-75% (w/w).

Sodium Carbomer 0.5 to 2.0% (w/w).

Carrageenan 0.5 to 2.0% (w/w).

Glycerin 10% to 40% (w/w).

Polyethylene Glycol 20% to 50% (w/w).

Hydroxyethyl (methyl) cellulose 0.5% to 2.0% (w/w).

In one embodiment, which is a preferred embodiment, the percentage weight (w/w) of the ingredients in the Gel/Lubricant is:

Water 50% (w/w).

Sodium Carbomer 1.0% (w/w).

Kappa Carrageenan 0.5% (w/w).

Glycerin 10% (w/w).

Polyethylene Glycol 20% (w/w).

In another embodiment, the percentage weight (w/w) of the ingredients in the gel/lubricant is:

Water 50% (w/w).

Sodium Carbomer 1.0% (w/w).

Kappa Carrageenan 0.5% (w/w).

Hydroxyethyl Methyl Cellulose 0.5% (w/w).

Polyethylene Glycol 20% (w/w).

PEG-3350 5% (w/w).

### D. Conventional Main Element: Ph Adjuster

A pH adjuster is a blend of ingredients that buffers the pH to a range between 3.2 and 10.6, most preferably at 4.0, or which value that most closely matches the pH of the mucous membranes in which the composition is applied

Citric acid or Acetic Acid combined with a less than molar equivalent of Sodium Hydroxide or Potassium Hydroxide is an example of a pH adjuster.

In producing the liquid compositions of the present disclosure, there are preferred amounts of pH adjusters. More particularly, it is preferred to use the following ingredients in the ranges of:

Sodium Citrate: 0.05% to 0.3% (w/w)

Potassium Acetate: 0.05% to 0.3% (w/w)

In one embodiment, which is a preferred embodiment, the percentage weight (w/w) of the pH adjuster in the mixture is

Potassium Acetate. 0.25% (w/w)

In another embodiment, the percentage weight (w/w) of the pH adjuster in the mixture is

Sodium Citrate. 0.25% (w/w)

### E. Conventional Main Element: Moisturizer

Water is the source of moisture.

Water always makes up the remaining percentage of the composition, and the preferred amount is between 25 to 75% (w/w).

In one embodiment, which is a preferred embodiment, the percentage of water is 50% (w/w).

In all embodiments, water is used to dissolve the solid ingredients in the composition that may include the gelling compound (Carbomer and Carrageenan and Cellulose), the ph adjuster (Citric Acid), and the preservative (Sodium Benzoate).

### F. Conventional Main Element: Preservative

A preservative is a substance that is added to the gel/lubricant to prevent decomposition by microbial growth or by undesirable chemical changes.

Preservatives may include sodium benzoate, methylparaben, propylparaben, or sodium ascorbate.

In producing the liquid compositions of the present disclosure, there are preferred amounts of preservatives. More particularly, it is preferred to use the following ingredients in the ranges of:

Methylparaben: 0.01% to 0.2% (w/w)

Propylparaben: 0.01% to 0.2% (w/w)

Sodium Benzoate: 0.01% to 0.4% (w/w)

In one embodiment, which is a preferred embodiment, 0.25% (w/w) of Sodium Benzoate is added as a preservative to the mixture.

In another embodiment, 0.05% (w/w) of methylparaben and 0.05% (w/w) of propylparaben is added as a preservative to the mixture.

### G. Conventional Main Element: Flavor

Natural and synthetic flavors are an optional conventional main element. Citrus flavor is an example. Of a flavor and its ingredient is citric acid.

In producing the liquid compositions of the present disclosure, there are preferred amounts of flavors. More particularly, it is preferred to use the following ingredients in the ranges of:

Citrus Flavor: 0.01% to 0.2% (w/w)

In one embodiment, which is a preferred embodiment, 0.075% (w/w) of Citric Acid is added to the mixture to impart a Citrus flavor.

### H. Conventional Main Element: Sweetener

Sweeteners are an optional conventional main element. Sweetness in the gel/lubricant composition can come from ingredients such as sorbitol, glycerin, xylitol, or other food grade sugar alcohols.

In producing the liquid compositions of the present disclosure, there are preferred amounts of sweeteners. More particularly, it is preferred to use the following ingredients in the ranges of:

Sweetener, such as sorbitol or xylitol 1.0% to 3.0% (w/w)

In one embodiment, which is a preferred embodiment, 2% (w/w) of the gel/lubricant contains sorbitol.

In another embodiment, 2% (w/w) of the gel/lubricant contains xylitol.

### I. Connections of Main Elements and Sub-Elements of Invention

The Personal Lubricant containing Volatile Fatty Acids is a liquid matrix that can be made by mixing all the Conventional Main Ingredients together, heating until all the ingredients are completely dissolved, then cooling this to room temperature, then adding in the Novel Main Ingredient, the Blend of Volatile Fatty Acids. The mixture is blended and distributed into plastic squeeze-bottle containers and labeled.

The process can be carried out by a batch process, a continuous process or a combination of the two. The order of addition of the ingredients can also be varied to yield acceptable product depending upon the types of ingredients being blended together and their respective physical characteristics.

The resulting gel/lubricant composition may be colored or colorless, flavored or flavorless, water-based, and/or may include humectants. The vaginal formula is preferably pH balanced with a range between 3.2 and 10.6, preferably 4.0. The viscosity range is between 200-30,000 centipoise, preferably 5,500 centipoise.

The liquid matrix is applied inside the vagina in an amount preferred by the users prior to intercourse to efficiently coat the vaginal canal. The odor and scent are released into the air to increase sexual intimacy prior to, during and/or after intercourse.

In one embodiment, which is a preferred embodiment, and is an example, the invention is made as follows:

1. Add 100 ml of distilled water into an empty a 500 ml beaker.

2. Add a stir bar to the beaker and begin stirring.

3. Heat water to 80 degrees C. over a period of 10-20 minutes

4. Add 0.8 grams of Kappa Carrageenan.

5. Add 1.0 grams of Sodium Carbomer.

6. Add. 31 grams of Potassium Hydroxide.

7. Add. 29 grams of Acetic Acid.

8. Add 1.0 grams of Sorbitol.

9. Add 0.15 grams of Citric Acid.

10. Add 40 ml of food grade Glycerin.

11. Add 55 ml of food grade Propylene Glycol.

12. Add 5.0 grams of PEG-3340.

13. Add 0.5 grams of Sodium Benzoate.

14. Cool the mixture to room temperature, optionally using an ice bath.

15. Add 0.016 grams of Butyric Acid.

16. Add 0.012 grams of Iso-Butyric Acid.

17. Add 0.008 grams of Iso-Valeric Acid.

18. Add 0.004 grams of Caproic Acid.

19. Blend mixture thoroughly, with vigorous aeration.

20. Transfer to plastic squeeze bottles and label.

In another embodiment, as an example, the invention is made as follows:

1. Add 100 ml of distilled water into an empty a 500 ml beaker.

2. Add a stir bar to the beaker and begin stirring.

3. Heat water to 80 degrees C. over a period of 10-20 minutes

4. Add 0.5 grams of Kappa Carrageenan.

5. Add 1.0 grams of Sodium Carbomer.

6. Add 0.5 grams of Hydroxyethyl Methyl Cellulose.

7. Add 0.31 grams of Potassium Hydroxide.

8. Add 0.29 grams of Acetic Acid.

9. Add 0.15 grams of Citric Acid.

10. Add 40 ml of food grade Glycerin.

11. Add 60 ml of food grade Propylene Glycol.

12. Add 0.5 grams of MethylParaben.

13. Cool the mixture to room temperature, optionally using an ice bath.

14. Add 0.008 grams of Butyric Acid.

15. Add 0.006 grams of Iso-Butyric Acid.

16. Add 0.004 grams of Iso-Valeric Acid.

17. Add 0.002 grams of Caproic Acid.

18. Blend mixture thoroughly, with vigorous aeration.

19. Transfer to plastic squeeze bottles and label.

In yet a further embodiment, as an example, the invention is made as follows:

1. Add 100 ml of distilled water into an empty a 500 ml beaker.

2. Add a stir bar to the beaker and begin stirring.

3. Heat water to 80 degrees C. over a period of 10-20 minutes

4. Add 2.0 grams of Sodium Carbomer.

5. Add 0.31 grams of Potassium Hydroxide.

6. Add 0.29 grams of Acetic Acid.

7. Add 1.0 grams of Sorbitol.

8. Add 0.15 grams of Citric Acid.

9. Add 100 ml of food grade Propylene Glycol.

10. Add 0.5 grams of Sodium Benzoate.

11. Cool the mixture, optionally using an ice bath.

12. Add 0.004 grams of Butyric Acid.

13. Add 0.003 grams of Iso-Butyric Acid.

14. Add 0.001 grams of Valeric Acid.

15. Add 0.001 grams of Iso-Valeric Acid.

16. Add 0.0005 grams of Caproic Acid.

17. Add 0.0005 grams of Iso-Caproic Acid.

18. Blend mixture thoroughly, with vigorous aeration.

19. Transfer to plastic squeeze bottles and label.

### J. Alternative Embodiments of Invention

Alternatively, a body lotion containing Volatile Fatty Acids rather than a gel/lubricant containing Volatile Fatty Acids is a variation of the invention.

### K. Operation of Preferred Embodiment

The liquid matrix is applied inside the vagina in an amount preferred by the users prior to intercourse to efficiently coat the vaginal canal. The odor and scent are released into the air from the volatile fatty acids to increase sexual intimacy prior to, during and/or after intercourse.

The composition may be applied to the mucous membranes and provide or add lubricity to these membranes such the oral, rectal or vaginal mucosa.

The scent and odor of the volatile fatty acids are generally noticeable as soon as the composition is applied, but also after drying on the surface in which applied, including but not limited to vaginal labia, fingers, pubic hair, or skin anywhere on the body, or in the case of silicone dolls, on any part of the doll.

The length of time the scent and odor last is generally longer than the composition remains wet, but not permanently, as the volatile fatty acids are volatile in nature and eventually disperse.

The liquid matrix can be made by mixing all the Conventional Main Ingredients together, heating until all the ingredients are dissolved, then cooling to room temperature and adding in the Novel Main Ingredient, a unique blend of Volatile Fatty Acids.

In one embodiment, which is a preferred embodiment, the composition of all the elements in the lubricate is:

50% (w/w) Distilled Water.

0.4% (w/w) Kappa Carrageenan.

0.5% (w/w) Sodium Carbomer.

0.25% (w/w) Potassium Acetate.

0.5% (w/w) Sorbitol.

0.075% (w/w) Citric Acid.

20% (w/w) Food grade Glycerin.

25% (w/w) Food grade Propylene Glycol.

2.5% (w/w) PEG-3340.

0.5% (w/w) Sodium Benzoate.

0.008% (w/w) Butyric Acid.

0.006% (w/w) Iso-Butyric Acid.

0.004% (w/w) Iso-Valeric Acid.

0.002% (w/w) Caproic Acid.

In another embodiment, the composition of all the elements in the lubricate is:

50% (w/w) Distilled Water.

0.25% (w/w) Kappa Carrageenan.

0.5% (w/w) Sodium Carbomer.

0.25% (w/w) Hydroxyethyl Methyl Cellulose.

0.25% (w/w) Potassium Acetate.

0.075% (w/w) Citric Acid.

20% (w/w) Food Grade Glycerin.

30% (w/w) Food Grade Propylene Glycol.

0.25% (w/w) MethylParaben.

0.004% (w/w) Butyric Acid.

0.003% (w/w) Iso-Butyric Acid.

0.002% (w/w) Iso-Valeric Acid.

0.001% (w/w) Caproic Acid.

In yet a further embodiment, the composition of all the elements in the lubricate is:

50% (w/w) Distilled water

1.0% (w/w) Sodium Carbomer.

0.25% (w/w) Potassium Acetate.

0.5% (w/w) Sorbitol.

0.075% (w/w) Citric Acid.

48% (w/w) Food grade Propylene Glycol.

0.25% (w/w) Sodium Benzoate.

0.0002% (w/w) Butyric Acid.

0.0015% (w/w) Iso-Butyric Acid.

0.0005% (w/w) Valeric Acid.

0.0005% (w/w) Iso-Valeric Acid.

0.00025% (w/w) Caproic Acid.

0.00025% (w/w) Iso-Caproic Acid.

What has been described and illustrated herein is a preferred embodiment of the invention along with some of its variations. The terms, descriptions and figures used herein are set forth by way of illustration only and are not meant as limitations. Those skilled in the art will recognize that many variations are possible within the spirit and scope of the invention in which all terms are meant in their broadest, reasonable sense unless otherwise indicated. Any headings utilized within the description are for convenience only and have no legal or limiting effect.

### INDEX OF ELEMENTS

10: Novel Main Element: Volatile Fatty Acids
11: Acetic Acid
12: Propionic Acid
13: Butyric Acid
14: Iso-butyric Acid
15: Valeric Acid
16: Iso-valeric Acid
17: Caproic Acid
18: Iso-caproic Acid
20: Conventional Main Element: Gel/Lubricant
21: Sodium Carbomer
22: Carrageenan
23: Hydroxyethyl Methyl Cellulose
24: Hydroxyethyl Cellulose
25: Glycerin
26: Propylene Glycol (PEG)
27: PEG-3350
30: Conventional Main Element: Ph adjuster
31: Sodium Citrate
32: Potassium Acetate
33: Sodium Acetate
34: Potassium Citrate Acid
40: Conventional Main Element: Moisturizer
41: Water
50: Conventional Main Element: Preservative
51: Sodium Benzoate
52: Methylparaben
53: Propylparaben
54: Sodium Ascorbate
60: Conventional Main Element: Flavor
61: Citric Acid
62: Natural Flavors
63: Synthetic Flavors
70: Conventional Main Element: Sweetener
71: Sorbitol
72: Glycerin
73: Xylitol
74: Sugar Alcohols

1. The personal lubricant contains an unique concentration and blend of the following volatile fatty acids:
a) acetic acid
b) propionic acid
c) butyric acid
d) iso-butyric acid
e) valeric acid
f) iso-valeric acid
g) caproic acid
a) iso-caproic acid
